(19) United States
(12) Patent Application Publication
(10) Pub. No.: US 2013/0030468 A1
(43) Pub. Date: Jan. 31, 2013

(54) TRANSVERSE CONNECTION SYSTEM AND DEVICE FOR THE VERTEBRAL COLUMN

(71) Applicants: Regis Le Couedic, Bordeaux (FR); Christian Baccelli, Saucats (FR)

(72) Inventors: Regis Le Couedic, Bordeaux (FR); Christian Baccelli, Saucats (FR)

(73) Assignee: IMPLANET, Martillac (FR)

(21) Appl. No.: 13/639,298

(22) PCT Filed: Apr. 5, 2011
(86) PCT No.: PCT/FR11/00200

§ 371 Date: Oct. 4, 2012

(30) Foreign Application Priority Data
Apr. 8, 2010 (FR) 1001489

(51) Int. Cl. A61B 17/70

(52) U.S. Cl. 606/250

(57) ABSTRACT

The invention relates to a connection system (3, 12, 13) between two longitudinal cylindrical spinal support rods (4), said connection system comprising at least one transverse elongate element (6, 14) having, on each end thereof, a head (7, 8; 17, 18; 43) for attaching onto a corresponding rod. At least one of said attachment heads (7, 8; 17, 18) includes a semi-cylindrical recess (19) for clipping with the rod (4), said recess having an upper lip (20, 21), covering the bottom of the rod, and a lower lip (22, 23), that is radially opposite said upper lip, and the head comprises a means (11, 24) for bending said lower lip (22, 23) between a position for connecting and a position for locking onto the rod.

The present invention relates to a connection system between two longitudinal, cylindrical spinal support rods, said connection system having at least one transverse elongate element provided, at each of its ends, with a head for fixing on a corresponding rod.

It also relates to a device comprising such a system.

It has an important application mainly, but not exclusively, in the repair of the vertebral column following trauma resulting from an accident, or for compensating for a malformation such as scoliosis or any other deformation, whether congenital or acquired.

It is customary in such cases for an implantable straightening and/or strengthening device to be fitted in place using two rods arranged substantially parallel to the vertebral column, on each side of the latter.

Each rod is fixed to at least two vertebrae, for example by screws that are screwed into a corresponding vertebra, or else by means of hooks.

Each screw or hook has a fixation part adjustable in position on the rod, the distance between the points of fixation on the adjacent vertebrae being intended to return the vertebrae to the desired position.

Such a device permits relief of the vertebral column. Its straightening and the presence of solid rods on each side, to which rods the vertebrae are attached, take up the pressure stresses exerted on the deformed or damaged vertebrae.

With such a device, it is often useful to provide a system for connecting the two parallel or substantially parallel rods to each other, which system permits better stiffening and strengthening of the assembly.

The invention relates to a system of this type.

Devices have already been disclosed for posterior fixation of the spine by pedicle screws, comprising at least two transverse bars that mechanically connect the two parallel longitudinal rods to the vertebral column.

WO 2009/117111, for example, proposes a device for fixing two spinal support rods to each other. The device comprises a transverse element provided, at each end, with a head for fixing on the corresponding rod. Each head comprises a removable bracket component for locking the rod introduced into a recess formed in the transverse element.

Each fixing head moreover comprises openworked chambers for penetration of an end of the element permitting horizontal and vertical clearance of the element, allowing it to adjust to the hollows and bumps of the column.

Such a device is awkward to fit in place and requires many small pieces to be screwed.

Systems are also known in which the fixation on the vertebrae is itself effected by way of the fixing heads of the transverse element.

Such systems have no flexibility and can function only in simple cases.

Generally, the systems of the prior art are not naturally stable, are awkward to use, take up a lot of space and do not permit really uniform clamping of the fixing component on the rod.

The aim of the present invention is to make available a connection system that meets the practical requirements better than the previously known systems, especially in that it permits an immediate hold of the connection element on the rods by clipping, which makes the system self-stabilizing and easier for the surgeon to fit in place, and in that it takes up very little space above the rod, amounting to the height of a head of a pedicle screw, which therefore avoids causing the patient muscular pain, and in that it permits perfectly uniform clamping of the fixing component on the rod, on a large surface area of the latter.

The system according to the invention is therefore indicated particularly for operations intended to reduce deformations (scoliosis), especially in young and/or slim patients with a low-volume thoracic area.

To this end, the invention basically proposes a connection system between two longitudinal, cylindrical spinal support rods, said connection system having at least one transverse elongate element provided, at each of its ends, with a head for fixing on a corresponding rod, characterized in that at least one of said fixing heads comprises a semi-cylindrical recess for clipping onto the rod, said recess having an upper lip, which covers the top of the rod, and a radially opposite lower lip, and in that said head has means of deformation by bending said lower lip between a position of connection and a position of locking on the rod.

The semi-cylindrical recess for clipping permits, in particular, a uniform clamping on the rod, since the surface of contact with the rod is in fact the internal surface of the recess with a cross section equal or substantially equal to half the circumference of the rod.

In advantageous embodiments, use is also made of one and/or more of the following arrangements:
the head is formed by a body comprising, on one side, said recess and, on the other side, the means for deformation of the lower lip;
the means of deformation have a slotted zone comprising a lower tongue rigidly connected to the lower lip and separated from the rest of the body by a slot, said rest of the body being rigidly connected to the elongate element, and means for spacing said tongue from the rest of the body in order to deform said lower lip between its position of connection and its locking position;
the spacing means are formed by a clamping screw, which bears at one end on the inner face of the slot of the tongue and cooperates at the other end with a thread integral with the rest of the body;
the two fixing heads have an identical configuration;
the recesses are directed to the same side with respect to a defined transverse direction;
the other head comprises a cylindrical recess for passage of one of said cylindrical rods and able to cooperate with said rod under gentle friction;
the transverse elongate element comprises two branches, each rigidly connected to a corresponding head and designed to slide relative to each other, and means for locking one of the branches on the other in the frontal and horizontal planes using a locking screw;
the locking screw cooperates with a thread belonging to an intermediate head rigidly connected to one of the two branches, said intermediate head comprising a recess for passage of the end of the other branch, said recess permitting play between the end and said intermediate head in the frontal and horizontal planes, the end of the screw for its part being designed to bear on and lock the other branch in the recess when it is compressed by screwing on the latter.

The invention also proposes a device comprising at least two cylindrical rods and at least one system of the kind described above.

The invention will be better understood on reading the following description of embodiments given as non-limiting examples. Reference is made in the description to the accompanying drawings, in which:

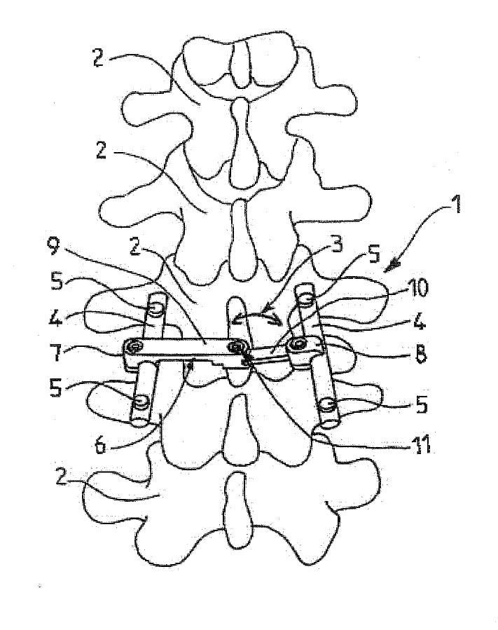

FIG. 1 is a front view of a portion of the vertebral column provided with a system for connection between two rods, according to a first embodiment of the invention.

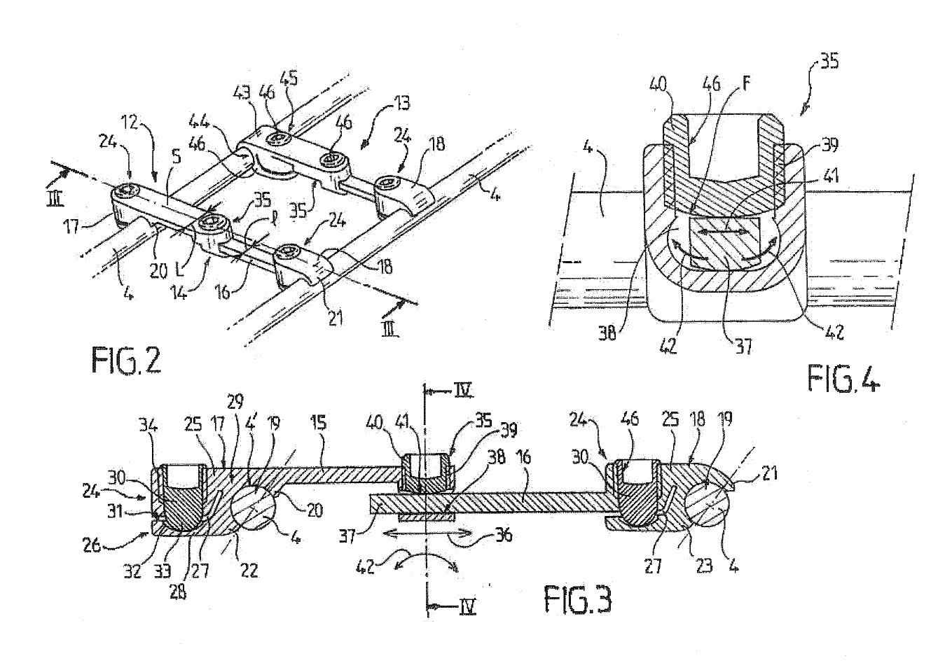

FIG. 2 is a partial perspective view of two rods provided with two connection systems, according to two embodiments of the invention.

FIG. 3 is a sectional view, along III-III, of one of the systems in FIG. 2.

FIG. 4 is a sectional view, along IV-IV, of the fixing head of the device in FIG. 3.

FIG. 1 shows a portion 1 of the vertebral column comprising vertebrae 2, and a connection system 3, according to one embodiment of the invention, between two cylindrical rods 4.

The rods are fixed on the vertebrae by pedicle screws 5 in a manner known per se.

The system 3 comprises a transverse elongate element 6 which is provided, at each of its ends, with a head 7 and 8 for fixing on a corresponding rod 2.

The elongate element 6 comprises two branches 9 and 10 rigidly connected to each other and designed to slide relative to each other, and means 11 for blocking one of the branches 9 relative to the other branch 10 in the frontal plane (parallel to the rods) and horizontal plane (perpendicular to the rods).

FIG. 2 is a perspective view of two embodiments 12 and 13 of the system according to the invention.

In the remainder of the text, the same reference numbers will be used to designate the same elements.

The system 12 comprises a transverse elongate element 14 composed of two branches 15 and 16, namely a first branch 15 comprising a head 17 for fixing on the rod 4, and a second branch 16 comprising a head 18 for fixing on the other rod 4.

Each fixing head (cf. FIG. 3) comprises a semi-cylindrical recess 19, which clips onto the rod and has an upper lip 20, 21 partially covering the top 4′ of the rod, and a radially opposite or substantially radially opposite lower lip 22, 23.

The recesses 19 and their lips are arranged to allow the component to be clipped onto a corresponding rod 4, by virtue of the possibility of slight deformation of the lip on the rigid rod.

According to one aspect of the invention, the lower lips 22, 23 can be deformed.

More precisely, each fixing head has means 24 for deformation of the lip 22, 23 by bending the latter between a free position of connection, which permits easy clipping, and a blocking position, in which the lip is pressed onto the rod.

To do this, the head is formed by a body 25 comprising, on one side, the recess 19 and, on the other side, the means 24 for deformation.

These comprise a slotted zone 26 with a transverse slot 27, which has a cross section in the shape of an L or of a hockey stick and separates a portion 28, in the shape of a lower flat tongue, from the rest 29 of the body which comprises, on one side, the recess 19 formed in the branch 15, on the same side for one of the heads 17 and on the other side for the head 18, and which comprises, on the other side, means for spacing said tongue apart from the rest of the body.

These spacing means are formed by a screw 30 of large diameter, for example 1 cm, with a rounded point, said rounded point bearing on the inner face 31, for example having a cup-shaped recess 32 for receiving the for example spherical end 33 of the screw 30 and cooperating, at the other end, with a thread 34 formed on the rest 29 of the body.

The branch 15 has an elongate portion with a first width L, for example parallelepipedal, and, at the end remote from the fixing head 17, terminates on the base 4 by way of an intermediate head 35, for example in the form of a cylindrical stub designed to allow the branch 16 to slide relative to the branch 15 in the transverse direction (arrow 36) with respect to the rods 4.

The branch 16, for its part, is terminated at one end by the fixing head 18.

It has an elongate portion which, for example, is parallelepipedal with a partially cylindrical or at least rounded concave bottom (cf. FIG. 4) and with a second width l<L and is connected, at the other end, to the rest of the body 29 of the head 18 at a portion situated opposite the rod 4 with respect to the spacing screw 30.

The other end 37 of the elongate portion passes through a recess 38 of the intermediate head 35, with which it cooperates with friction.

The head 35 has a threaded orifice 39 and a locking screw 40, which is of the same type as the spacing screw 30 and is provided with a spherical end 41 that will permit flexibility of the branch 16 with respect to the branch 15 according to the arrow 42 in the transverse vertical plane.

The head 35 is described in more detail with reference to FIG. 4.

The head 35 is recessed in the lower part with a recess 38 whose cross section is substantially oval or in the shape of a crushed circle and is wider, for example one and a half times wider, than the width l of the branch, which will permit a lateral movement (arrow 41) of said branch 16 relative to the head 35 rigidly connected to the branch 15, and a possibility of pivoting about the transverse axial direction of the branches (arrow 42).

Similarly, the rounded bottom F of the screw 40 permits angular clearance in the plane perpendicular to the branches.

The junction between the two branches thus makes it possible to compensate for the possible angular offsets between the two rods of union on the one hand and in the plane parallel to the vertebrae on the other hand, permitting a slight twisting of one branch relative to the other.

FIG. 2 shows another embodiment of the system 13 of the invention, for which the fixing head 43 on one of the rods is different.

Here, the head 43 comprises a cylindrical orifice or recess 44 for passage of the rod, which will thus be introduced beforehand, means 45 for compressing the lower part 46 of the edge of the orifice, of the type described with reference to the means 24, being provided, with slot, tongue and corresponding screw.

We will now describe the placement of a device using a system according to the embodiment of the invention with reference to FIGS. 1 and 3.

After the dorsal region of the patient has been opened up in order to gain access to the vertebrae that are to be straightened, the surgeon fits the rods in place by fixing them in a manner known per se, for example by screwing the pedicle screws into the ends of the vertebrae concerned.

He is then able, in an extremely simple and quick way, to fit in place the connection system, for example made of titanium or biocompatible material, with dimensions permitting a certain degree of flexibility.

The central head being movable (screw 40 unscrewed), he then clips the system in place, which then has the necessary degrees of freedom in the frontal and sagittal planes (arrows 36 and 42) to permit the clipping on each side without constraints.

He then screws the fixing screws 30 from above onto the rods, which has, for example, heads with a hexagonal socket 46 for screwing in a manner known per se.

This permits final locking on the rods 4.

Finally, the assembly is stiffened by adjustment and screwing of the central screw 40 of the head 35.

The surgeon then fits in place as many stiffening systems 3 as he deems necessary.

The system is advantageously made of titanium alloy, but it can also be produced from other biocompatible materials having good characteristics of elasticity.

It goes without saying, and it is also obvious from the above, that the present invention is not limited to the embodiments that have been more particularly described. On the contrary, it encompasses all variants thereof, especially those in which the branches are cylindrical, those in which the intermediate head is removable and/or those in which the two fixing heads are identical but situated between the two rods, in an opposite direction in relation to each other.

1. A connection system (3, 12, 13) between two longitudinal, cylindrical spinal support rods (4), said connection system having at least one transverse elongate element (6, 14) provided, at each of its ends, with a head (7, 8; 17, 18; 43) for fixing on a corresponding rod, at least one of said fixing heads (7, 8; 17, 18) comprising a semi-cylindrical recess (19) for clipping onto the rod (4), said recess having an upper lip (20, 21) and a radially opposite lower lip (22, 23), said head having means (11, 24) of deformation by bending said lower lip (22, 23) between a position of connection and a position of locking on the rod, characterized in that the upper lip covers the top of the rod,
in that the recesses (19) are directed to the same side with respect to a defined transverse direction,
in that the transverse elongate element comprises two branches (15, 16), each rigidly connected to a corresponding head (17, 18) and designed to slide relative to each other, and means (35) for locking one of the branches (15) on the other (16) in the frontal and horizontal planes using a locking screw (40),
and in that the locking screw cooperates with a thread belonging to an intermediate head rigidly connected to one of the two branches, said intermediate head comprising a recess for passage of the end of the other branch, said recess permitting play between the end and said intermediate head in the frontal and horizontal planes, the end of the screw for its part being designed to bear on and lock the other branch in the recess when it is compressed by screwing on the latter.

2. The system as claimed in claim 1, characterized in that the head (17, 18) is formed by a body (25) comprising, on one side, said recess (19) and, on the other side, the means (24) for deformation of the lower lip.

3. The system as claimed in claim 2, characterized in that the means (24) of deformation have a slotted zone (26) comprising a lower tongue (28) rigidly connected to the lower lip (22, 23) and separated from the rest (29) of the body by a slot (27), said rest (29) of the body being rigidly connected to the elongate element, and means (30, 31) for spacing said tongue from the rest of the body in order to deform said lower lip between its position of connection and its locking position.

4. The system as claimed in claim 3, characterized in that the spacing means (30, 31) are formed by a clamping screw (30), which bears at one end on the inner face (31) of the slot of the tongue and cooperates at the other end with a thread (34) integral with the rest (29) of the body.

5. The system as claimed in claim 1, characterized in that the two fixing heads (17, 18) have an identical configuration.

6. The system as claimed in claim 1, characterized in that the other head comprises a cylindrical recess (44) for passage of one of said cylindrical rods (4) and able to cooperate with said rod under gentle friction.

7. A device characterized in that it has two cylindrical rods (4) and at least one system (3, 12, 13) as claimed in claim 1.
